(19) United States
(12) Patent Application Publication
(10) Pub. No.: US 2026/0210846 A1
(43) Pub. Date: Jul. 23, 2026

(54) METHOD AND SYSTEM FOR IMPROVING THE ACCURACY OF SATELLITE REMOTE SENSING INVERSION BY INTEGRATING HYPERSPECTRAL PROXIMAL SENSING

(71) Applicants: Nanjing Institute of Geography and Limnology, Chinese Academy of Sciences, Nanjing City (CN); Nanjing Zhongke Deep Insight Technology Research Institute Co., Ltd., Nanjing City (CN)

(72) Inventors: Na LI, Nanjing City (CN); Yunlin ZHANG, Nanjing City (CN); Yibo ZHANG, Nanjing City (CN); Kun SHI, Nanjing City (CN); Xiayang LUO, Nanjing City (CN)

(21) Appl. No.: 19/428,038

(22) Filed: Dec. 19, 2025

(30) Foreign Application Priority Data
Jan. 20, 2025 (CN) 2025100805244

(51) Int. Cl. G01N 21/31; G01N 33/18

(52) U.S. Cl. CPC G01N 21/31; G01N 33/18; G01N 2201/1293

(57) ABSTRACT

The present disclosure discloses a method and system for improving the accuracy of satellite remote sensing inversion by integrating hyperspectral proximal sensing, belonging to the technical field of water environment and aquatic ecological monitoring. The method includes the steps of: acquiring a water body hyperspectral proximal sensing reflectance spectrum dataset, a satellite remote sensing surface reflectance dataset, and a water environment in-situ measurement dataset, and performing preprocessing on the datasets; generating a fused satellite-ground dataset, and constructing and training a water quality parameter inversion model; and updating corresponding methods and the datasets in real time by repeating specified steps, thereby optimizing the model. The present disclosure displays and downloads inversion results according to a query instruction, enhances satellite spectral precision and increases the scale of the synchronous satellite-ground dataset, and is used for high-precision water environment monitoring.

### CROSS-REFERENCE TO RELATED APPLICATIONS

This application claims priority to Chinese Patent Application No. 202510080524.4, filed on Jan. 20, 2025, which is incorporated herein by reference in its entirety.

### TECHNICAL FIELD

The present disclosure pertains to the technical field of water environment and aquatic ecological monitoring, and specifically relates to a method and system for improving the accuracy of satellite remote sensing inversion by integrating hyperspectral proximal sensing.

### BACKGROUND

Traditional water quality monitoring primarily relies on manual sampling and laboratory measurements, which is time-consuming, labor-intensive, and struggles to meet the timeliness requirements for large-scale water quality monitoring, especially for rapidly changing water bodies. Satellite remote sensing is widely used for water environment inversion and monitoring due to its advantages of large-scale coverage, periodicity, and cost-effectiveness. However, affected by weather conditions and incomplete atmospheric correction, the accuracy of satellite-acquired spectral data still requires further enhancement. Furthermore, while satellite remote sensing features large-scale instantaneous acquisition, river and lake water quality exhibits highly dynamic characteristics. Consequently, obtaining spatiotemporally consistent quasi-synchronous satellite-ground data is challenging, and ground-based data within 3 hours before or after a satellite overpass are typically used for matching.

Moreover, the accuracy and generalizability of water quality remote sensing inversion models depend on the quantity and diversity of the quasi-synchronous satellite-ground dataset. However, the acquisition of synchronous ground data with substantial diversity is constrained by ground sampling personnel and costs, making it difficult to obtain a large number of strictly diverse synchronous water samples. In summary, current water quality monitoring using satellite remote sensing inversion still faces challenges including insufficient atmospheric correction accuracy, temporal inconsistency between satellite and ground data, and limitations in both the data volume and diversity of strictly synchronous satellite-ground datasets.

### SUMMARY

The objective of the present disclosure is to address the aforementioned problems by providing a method and system for improving the accuracy of satellite remote sensing inversion by integrating hyperspectral proximal sensing.

The present disclosure adopts the following technical solution: A method for improving the accuracy of satellite remote sensing inversion by integrating hyperspectral proximal sensing includes the steps of:
Step 1: acquiring a water body hyperspectral proximal sensing reflectance spectrum dataset, a satellite remote sensing surface reflectance dataset, and a water environment in-situ measurement dataset, and selecting corresponding data preprocessing methods to perform preprocessing;
Step 2: determining an optimal matching window by using a temporal matching principle and constructing a synchronous dataset; wherein the synchronous dataset includes: synchronous data of SRrs(λ) and water quality, and synchronous data of fnR(λ) and water quality; and wherein the SRrs(λ) is preprocessed satellite remote sensing surface reflectance data, the fnR(λ) is preprocessed water body hyperspectral proximal sensing reflectance spectrum data, and λ is a band;
Step 3: creating a method for simulating multispectral data from hyperspectral data, generating corresponding synchronous data of nGRrs(λ) and water quality based on the synchronous data of fnR(λ) and water quality, and obtaining synchronous data of SRrs(λ) and nGRrs(λ), wherein the nGRrs(λ) is simulation-derived satellite remote sensing surface reflectance;
Step 4: performing normalization processing on the preprocessed satellite remote sensing surface reflectance data SRrs(λ) based on the synchronous data of SRrs(λ) and nGRrs(λ) to obtain new satellite remote sensing surface reflectance data nSRrs(λ), performing a comparative analysis between the new satellite remote sensing surface reflectance data nSRrs(λ) and the simulation-derived satellite remote sensing surface reflectance nGRrs(λ), and establishing a quantitative conversion relationship between nSRrs(λ) and nGRrs(2) band-by-band to eliminate error;
Step 5: combining the synchronous data of nGRrs(λ) and water quality and the synchronous data of nSRrs(λ) and water quality to generate a fused satellite-ground dataset; and using the synchronous data of SRrs(λ) and water quality and the fused satellite-ground dataset as input features, respectively, and using the water environment in-situ measurement dataset as an output feature, to construct and train a water quality parameter inversion model; and
Step 6: repeating Step 3 to Step 5, updating the method for simulating multispectral data from hyperspectral data, a method for enhancing satellite spectral band precision, and the fused satellite-ground dataset in real time, continuously optimizing the water quality parameter inversion model, and displaying and downloading inversion results according to a query instruction until a query termination instruction from a user is received.

In a further embodiment, data preprocessing steps for the water body hyperspectral proximal sensing reflectance spectrum dataset include:
continuously collecting data under various weather conditions to obtain the water body hyperspectral proximal sensing reflectance spectrum dataset {R(λ)}, and calculating a ratio of a water body hyperspectral proximal sensing reflectance spectrum R(λ) for each band to an average hyperspectral proximal sensing reflectance R₀ within 420 nm to 830 nm, to obtain a normalized hyperspectral proximal sensing reflectance nR(λ) corresponding to the band:

$$n R ( λ ) = \frac{R ( λ )}{{R}_{0}} ;$$

performing radiometric correction on nR(λ) with reference to spectral data of a synchronous terrestrial object spectrum collected by an Analytical Spectral Devices (ASD) Field spectrometer, with the calculation formula being:

$$f n R ( λ ) = α \times n R ( λ ) + b ;$$

where a is a radiometric gain value between the spectrum of Analytical Spectral Devices (ASD) Field spectrometer and water body hyperspectral proximal sensing reflectance sensor, and b is a radiometric offset value between the spectrum of Analytical Spectral Devices (ASD) Field spectrometer and water body hyperspectral proximal sensing reflectance sensor.

In a further embodiment, data preprocessing steps for the satellite remote sensing surface reflectance dataset include:
acquiring the satellite remote sensing surface reflectance dataset {SR(λ)}, extracting a quality band qa60, and obtaining a cloud mask file pixel value mask_{cloud} according to a bitwise operation principle;
generating an opaque cloud mask file and a cirrus cloud mask file respectively by using binary bitwise operations and the quality band qa60, merging the opaque cloud mask file and the cirrus cloud mask file using an AND( ) function to generate the cloud mask file, thereby obtaining the cloud mask file pixel value mask_{cloud}, and
processing with the following formula to obtain the preprocessed satellite remote sensing surface reflectance data SRrs(λ):

$$S R r s ( λ ) = \frac{S R ( λ )}{π \times 10000} \times m a s {k}_{c l o u d} ,$$

where SR(λ) is satellite remote sensing surface reflectance.

In a further embodiment, the steps for determining the optimal matching window include:
defining a sum of coefficients of determination for all bands as S for screening out an optimal temporal matching window, with the calculation formula being:

$$S = {R}_{443}^{2} + {R}_{490}^{2} + {R}_{560}^{2} + {R}_{665}^{2} + {R}_{705}^{2} + {R}_{740}^{2} + {R}_{783}^{2} ;$$

wherein

$${R}_{443}^{2} and {R}_{490}^{2} , {R}_{560}^{2} , {R}_{665}^{2} , {R}_{705}^{2} , {R}_{740}^{2} , {R}_{783}^{2}$$

are coefficients of determination for bands λ∈[443, 490, 560, 665, 705, 740, 783], respectively; and selecting a temporal matching window corresponding to a maximum value of S as the optimal matching window.

In a further embodiment, the method for simulating multispectral data from hyperspectral data is represented by the following formula:

$$n G R r s ( λ ) = \frac{\int fnR ( λ ) \times S ( λ ) d λ}{\int S ( λ ) d λ} , λ ∈ [ 443 , 490 , 560 , 665 , 705 , 740 , 783 ] ;$$

where fnR(λ) is the preprocessed water body hyperspectral proximal sensing reflectance spectrum data, and S(λ) is a satellite sensor spectral response function at λnm.

In a further embodiment, a calculation formula for the normalization processing of the preprocessed satellite remote sensing surface reflectance data SRrs(λ) is as follows:

$$n S R r s ( λ ) = \frac{SRr s ( λ )}{\begin{matrix}( SRrs ( 443 ) + S R r s ( 490 ) + S R r s ( 560 ) + \\ SRrs ( 66 \; 5 ) + S R r s ( 705 ) + S R r s ( 740 ) + S R r s ( 783 ) ) / 7\end{matrix}} ;$$

wherein λ∈[443, 490, 560, 665, 705, 740, 783], SRrs(443) represents satellite remote sensing surface reflectance data for a band of 443 nm, and likewise for the remaining bands.

In a further embodiment, the quantitative conversion relationship between nSRrs(λ) and nGRrs(λ) is expressed as follows:

$$n G R r s ( λ ) = slop \times nSRrs ( λ ) + c ;$$

wherein, slop is a linear regression slope, representing a gain situation between nSRrs(λ) and nGRrs(λ), and c is a constant.

In a further embodiment, elimination steps for the eliminating error include:
establishing an error analysis principle based on the linear regression slope slop, judging an error situation between nSRrs(λ) and nGRrs(λ) based on the error analysis principle, and performing targeted optimization according to the error situation;
wherein the error analysis principle is:

$$\{ \begin{matrix}\begin{matrix}slop < 1 & nSRrs ( λ ) is overestimated compared to nGRrs ( λ )\end{matrix} \\ \begin{matrix}slop = 1 & nSRrs ( λ ) and nGRrs ( λ ) have consistent response\end{matrix} \\ \begin{matrix}slop > 1 & nSRrs ( λ ) is underestimated compared to nGRrs ( λ )\end{matrix}\end{matrix} ;$$

and
further optimizing a calibration accuracy of a satellite sensor for water bodies when the overestimation or underestimation occurs.

In a further embodiment, the water quality parameter inversion model employs a machine learning model, and the machine learning model at least includes: a deep neural network model, a random forest model, a support vector machine model, an extreme gradient boosting tree model, a Gaussian process regression model, and a back-propagation neural network model.

A system for improving the accuracy of satellite remote sensing inversion by integrating hyperspectral proximal sensing, configured to implement the aforementioned method for improving the accuracy of satellite remote sensing inversion by integrating hyperspectral proximal sensing, wherein the system includes:
a proximal hyperspectral sensor, a satellite sensor, and a water quality detector, configured to acquire a water body hyperspectral proximal sensing reflectance spectrum dataset, a satellite remote sensing surface reflectance dataset, and a water environment in-situ measurement dataset, respectively;
a first module, configured to select corresponding data processing methods to perform data preprocessing on the water body hyperspectral proximal sensing reflectance spectrum dataset, the satellite remote sensing surface reflectance dataset, and the water environment in-situ measurement dataset, respectively;
a second module, configured to determine an optimal matching window by using a temporal matching principle and construct a synchronous dataset; wherein the synchronous dataset comprises: synchronous data of SRrs(λ) and water quality, and synchronous data of fnR(λ) and water quality; and wherein the SRrs(λ) is preprocessed satellite remote sensing surface reflectance data, the fnR(λ) is preprocessed water body hyperspectral proximal sensing reflectance spectrum data, and λ is a band;
a third module, configured to create a method for simulating multispectral data from hyperspectral data, generate corresponding synchronous data of nGRrs(λ) and water quality based on the synchronous data of fnR(λ) and water quality, and obtain synchronous data of SRrs(λ) and nGRrs(λ), wherein the nGRrs(λ) is simulation-derived satellite remote sensing surface reflectance;
a fourth module, configured to perform normalization processing on the preprocessed satellite remote sensing surface reflectance data SRrs(λ) based on the synchronous data of SRrs(λ) and nGRrs(λ) to obtain new satellite remote sensing surface reflectance data nSRrs(λ), perform a comparative analysis between the new satellite remote sensing surface reflectance data nSRrs(λ) and the simulation-derived satellite remote sensing surface reflectance nGRrs(λ), and establish a quantitative conversion relationship between nSRrs(λ) and nGRrs(2) band-by-band to eliminate error;
a fifth module, configured to combine the synchronous data of nGRrs(λ) and water quality and the synchronous data of nSRrs(λ) and water quality to generate a fused satellite-ground dataset; and use the synchronous data of SRrs(λ) and water quality and the fused satellite-ground dataset as input features, respectively, and use the water environment in-situ measurement dataset as an output feature, to construct and train a water quality parameter inversion model;
a sixth module, configured to repeat steps of the third module through the fifth module, update the method for simulating multispectral data from hyperspectral data, a method for enhancing satellite spectral band precision, and the fused satellite-ground dataset in real time, and continuously optimize the water quality parameter inversion model; and
a terminal display module, configured to display and download inversion results according to a query instruction until a query termination instruction from a user is received.

The present disclosure provides the following beneficial effects: The present disclosure presents users with a method for enhancing the accuracy of satellite-based water quality remote sensing inversion using near-ground hyperspectral data, providing an optimal time window for matching satellite spectral data with in-situ water quality measurements. This ensures a complete one-to-one correspondence between the spectral data and the in-situ water quality data obtained by the user.

The present disclosure expands the scale of synchronous satellite and ground in-situ measurement data through a simple and easy-to-operate simulation based on hyperspectral proximal sensing data. This increases the diversity and complexity of modeling data, ensures model stability and robustness, reduces the difficulty for users in obtaining synchronous satellite-ground datasets, and improves model accuracy.

The present disclosure enables the updating, querying, statistical analysis, uploading, and downloading of water quality information, ensuring users have access to water quality information across multiple temporal and spatial scales to assist in decision-making. Compared to traditional manual monitoring, which suffers from information lag, spatiotemporal discreteness, and a lack of long-term and multi-spatial variation characteristics, the method of the present disclosure provides a more convenient and efficient method and system.

The present disclosure supports interactive queries via a web interface, assisting users in obtaining water quality information of specific interest without interference from other information.

### BRIEF DESCRIPTION OF THE DRAWINGS

FIG. 1 is a flowchart of the method for improving the accuracy of satellite remote sensing inversion by integrating hyperspectral proximal sensing according to Embodiment 1.

FIG. 2 is a spectral response function diagram for the simulation of hyperspectral conversion to multispectral data according to Embodiment 1.

FIG. 3 is an analysis diagram of the consistency between synchronous SRrs(λ) and nGRrs(λ) according to Embodiment 1.

FIG. 4 is a diagram of the quantitative conversion relationship between nSRrs(λ) and nGRrs(λ) according to Embodiment 1.

FIG. 5 is a schematic diagram of a chlorophyll-a inversion model according to Embodiment 1.

### DETAILED DESCRIPTION OF THE EMBODIMENTS

The following further describes the present disclosure with reference to the accompanying drawings and embodiments.

### Embodiment 1

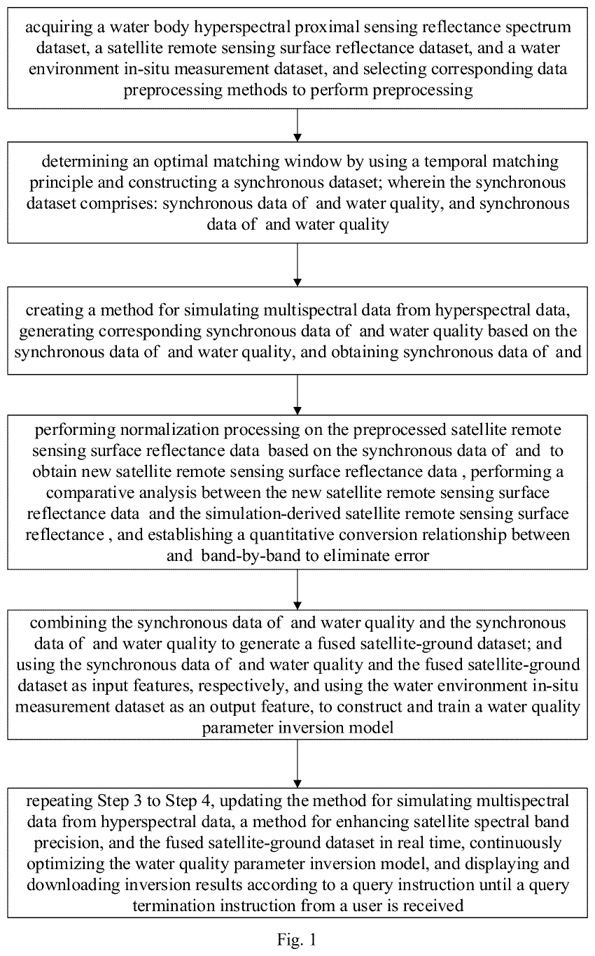

Given the complex and diverse nature of inland water bodies, and in order to improve satellite spectral precision, enhance the temporal matching consistency between satellite and ground spectra, and increase both the quantity and diversity of synchronous satellite-ground data, this embodiment provides a method for improving the accuracy of satellite remote sensing inversion by integrating hyperspectral proximal sensing (hereinafter referred to as “the method”). As shown in FIG. 1, the method includes the steps of:
Step 1: acquiring a water body hyperspectral proximal sensing reflectance spectrum dataset, a satellite remote sensing surface reflectance dataset, and a water environment in-situ measurement dataset, and selecting corresponding data preprocessing methods to perform preprocessing;
Step 2: determining an optimal matching window by using a temporal matching principle and constructing a synchronous dataset; wherein the synchronous dataset includes: synchronous data of SRrs(λ) and water quality, and synchronous data of fnR(λ) and water quality; and wherein the SRrs(λ) is preprocessed satellite remote sensing surface reflectance data, the fnR(λ) is preprocessed water body hyperspectral proximal sensing reflectance spectrum data, and λ is a band;
Step 3: creating a method for simulating multispectral data from hyperspectral data, generating corresponding synchronous data of nGRrs(λ) and water quality based on the synchronous data of fnR(λ) and water quality, and obtaining synchronous data of SRrs(λ) and nGRrs(λ), wherein the nGRrs(λ) is simulation-derived satellite remote sensing surface reflectance;
Step 4: performing normalization processing on the preprocessed satellite remote sensing surface reflectance data SRrs(λ) based on the synchronous data of SRrs(λ) and nGRrs(λ) to obtain new satellite remote sensing surface reflectance data nSRrs(λ), performing a comparative analysis between the new satellite remote sensing surface reflectance data nSRrs(λ) and the simulation-derived satellite remote sensing surface reflectance nGRrs(λ), and establishing a quantitative conversion relationship between nSRrs(λ) and nGRrs(λ) band-by-band to eliminate error;
Step 5: combining the synchronous data of nGRrs(λ) and water quality and the synchronous data of nSRrs(λ) and water quality to generate a fused satellite-ground dataset; and using the synchronous data of SRrs(λ) and water quality and the fused satellite-ground dataset as input features, respectively, and using the water environment in-situ measurement dataset as an output feature, to construct and train a water quality parameter inversion model; and
Step 6: repeating Step 3 to Step 5, updating the method for simulating multispectral data from hyperspectral data, a method for enhancing satellite spectral band precision, and the fused satellite-ground dataset in real time, continuously optimizing the water quality parameter inversion model, and displaying and downloading inversion results according to a query instruction until a query termination instruction from a user is received.

The following provides a detailed description of Steps 1 to 6. In this embodiment, the water body hyperspectral proximal sensing reflectance spectrum dataset, the satellite remote sensing surface reflectance dataset, and the water environment in-situ measurement dataset all comprise fundamental data information, such as the latitude and longitude of the collection point, collection time (which can be precise to the minute), type of water body collected, and weather conditions, among others. Furthermore, the weather conditions include: clear skies, overcast, cloudy, and rainy, and also include: wind speed magnitude, and so on.

Based on the aforementioned fundamental data information, the water body hyperspectral proximal sensing reflectance spectrum dataset is obtained as the ratio of upwelling to downwelling irradiance above the water surface, collected by a proximal hyperspectral sensor at a height of 2 to 10 meters above the water surface. The proximal hyperspectral sensor at least includes two types of monitoring equipment: a field spectroradiometer and a Ground-Based Proximal Sensing system (GBPSs). The satellite remote sensing surface reflectance dataset is Level 2 (L2) surface reflectance covering the visible to near-infrared bands acquired by a satellite-borne platform and subjected to radiometric correction, geometric correction, and atmospheric correction. Finally, the water environment in-situ measurement dataset at least includes optically active parameters such as chlorophyll-a, total suspended solids concentration, transparency, diffuse attenuation coefficient, and colored dissolved organic matter absorption coefficient.

To further exemplify, the satellite sensor in this embodiment is selected as Sentinel-2, the proximal hyperspectral sensor employs a GBPSs, and the water quality parameter selected is chlorophyll-a.

Based on the foregoing description, and considering the collection environments and the differences in collection parameters for the water body hyperspectral proximal sensing reflectance spectrum dataset, the satellite remote sensing surface reflectance dataset, and the water environment in-situ measurement dataset, this embodiment employs distinct data preprocessing methods for the water body hyperspectral proximal sensing reflectance spectrum dataset, the satellite remote sensing surface reflectance dataset, and the water environment in-situ measurement dataset. Specifically, the data preprocessing steps for the water body hyperspectral proximal sensing reflectance spectrum dataset include:

Continuously collecting data under various weather conditions to obtain the water body hyperspectral proximal sensing reflectance spectrum dataset {R(λ)}. To avoid inconsistencies in the hyperspectral proximal sensing reflectance caused by solar elevation, light intensity, and water surface waves in complex light environments, normalization processing is performed on the water body hyperspectral proximal sensing reflectance spectrum R(λ). Therefore, a ratio of the water body hyperspectral proximal sensing reflectance spectrum R(λ) for each band to an average hyperspectral proximal sensing reflectance R₀ within the 420 nm to 830 nm range is calculated to obtain a normalized hyperspectral proximal sensing reflectance nR(λ) corresponding to the band:

$$n R ( λ ) = \frac{R ( λ )}{{R}_{0}} ;$$

To further enhance the accuracy and reliability of the proximal hyperspectral bands, radiometric correction is performed on nR(λ) with reference to spectral data of a synchronous terrestrial object spectrum, with the calculation formula being:

$$f n R ( λ ) = α \times n R ( λ ) + b ;$$

where a is a radiometric gain value of a land-based hyperspectral proximal sensor, and b is a radiometric offset value of the land-based hyperspectral proximal sensor. In this embodiment, the value of a is 1.086, and the value of b is 0.0054.

It is worth mentioning that the formula for calculating the average hyperspectral proximal sensing reflectance R₀ mentioned above is as follows:

$${R}_{0} = \frac{R ( 420 ) + R ( 421 ) + R ( 422 ) + … + R ( 828 ) + R ( 829 ) + R ( 830 )}{830 - 420} ;$$

where R (420) is the water body hyperspectral proximal sensing reflectance for the 420 nm band, and so forth up to R(830).

Correspondingly, the data preprocessing steps for the satellite remote sensing surface reflectance dataset include:
acquiring the satellite remote sensing surface reflectance dataset {SR(λ)}, extracting a quality band qa60, and obtaining a cloud mask file pixel value mask cloud according to a bitwise operation principle;
generating an opaque cloud mask file and a cirrus cloud mask file respectively by using binary bitwise operations and the quality band qa60, merging the opaque cloud mask file and the cirrus cloud mask file using an AND( ) function to generate the cloud mask file, thereby obtaining the cloud mask file pixel value mask_{cloud}.

In a further embodiment, a method for generating the opaque cloud mask file includes: shifting the binary bit 1 left by 10 bits, then comparing the 10th bit (the 11th bit from the right) of the resulting binary value after the bitwise shift with the 10th bit (the 11th bit from the right) of the qa60 band value (in its binary form): if the two are identical, it indicates that the pixel is covered by thick cloud, and the pixel value is assigned 1; if the two are different, the pixel value is assigned 0, thereby ultimately generating the opaque cloud mask file.

Correspondingly, the steps for generating the cirrus cloud mask file include: shifting the binary bit 1 left by 11 bits, then comparing the 11th bit (the 12th bit from the right) of the resulting binary value after the bitwise shift with the 11th bit (the 12th bit from the right) of the qa60 band value (in its binary form): if the two are identical, it indicates that the pixel is covered by thin cloud, and the pixel value is assigned 1; if the two are different, the pixel value is assigned 0, thereby ultimately generating the cirrus cloud mask file.

Using the opaque cloud mask file and the cirrus cloud mask file obtained by the aforementioned method, the AND( ) function is used to merge them to obtain the cloud mask file, with corresponding value assignment to obtain the cloud mask file pixel value mask cloud. The specific code is expressed as follows:

$$qa 60. bitwiseAnd ( 1 << 10 ) . eq ( 0 ) . and ( qa60 . bitwiseAnd ( 1 << 11 ) . eq ( 0 ) ) .$$

Processing with the following formula to obtain the preprocessed satellite remote sensing surface reflectance data SRrs(λ):

$$S R r s ( λ ) = \frac{S R ( λ )}{π \times 10000} \times {mask}_{c l o u d} ,$$

where SR(λ) is satellite remote sensing surface reflectance.

Furthermore, data preprocessing methods for the water environment in-situ measurement dataset include: cleaning anomalous data below zero. Specifically, negative and zero values in laboratory-measured chlorophyll-a concentrations are deleted as outliers. Additionally, to further eliminate data anomalies caused by non-standard practices during manual sampling, field sampling photographs are compared with laboratory-measured chlorophyll-a concentrations. Data should be excluded when chlorophyll-a concentration is significantly low despite an increase in algal bloom intensity.

Traditionally, ground-based data from ±3 h to ±7 d around the satellite sensor overpass time are typically used for matching to obtain a sufficient volume of synchronous satellite-ground data. However, as water quality is dynamically changing due to climatic and watershed runoff influences, temporal inconsistency between satellite data and ground-based data acquisition can lead to mismatches between water body spectra and satellite spectra, which directly affects the accuracy and stability of the constructed water quality inversion model.

Further, with the satellite sensor overpass time as the temporal reference, water body hyperspectral proximal sensing reflectance are extracted for time windows of 0 min, ±1 h, ±2 h, ±3 h, ±1 d, ±2 d, and ±3 d, respectively, where “h” denotes hour and “d” denotes day. Matching is performed according to spatial and spectral criteria, and the coefficient of determination

$${R}_{λ}^{2}$$

for the λ nm band under different temporal matching windows is calculated. The calculation results are as follows:

$${R}_{λ}^{2} \geq 0.5$$

High consistency between satellite remote sensing surface reflectance and water body hyperspectral proximal sensing reflectance sensing reflectance

$${R}_{λ}^{2} < 0.5$$

Low consistency between satellite remote sensing surface reflectance and water body hyperspectral proximal
where λ∈[443, 490, 560, 665, 705, 740, 783].

Therefore, it is necessary to further select the optimal matching window for satellite-ground matching. The process for determining the optimal matching window in Step 2 by using the temporal matching principle is as follows: defining a sum of coefficients of determination for all bands as S for screening out an optimal temporal matching window, with the calculation formula being:

$$S = {R}_{443}^{2} + {R}_{490}^{2} + {R}_{560}^{2} + {R}_{665}^{2} + {R}_{705}^{2} + {R}_{740}^{2} + {R}_{783}^{2} ;$$

wherein

$${R}_{443}^{2} and {R}_{490}^{2} , {R}_{560}^{2} , {R}_{665}^{2} , {R}_{705}^{2} , {R}_{740}^{2} , {R}_{783}^{2}$$

are coefficients of determination for bands λ∈[443, 490, 560, 665, 705, 740, 783], respectively; and selecting a temporal matching window corresponding to a maximum value of S as the optimal matching window.

Taking chlorophyll-a as an example, and using the optimal matching window obtained via the aforementioned method, 77 pairs of synchronous data between SRrs(λ) and chlorophyll-a, and 1736 pairs of synchronous data between fnR(λ) and chlorophyll-a are obtained.

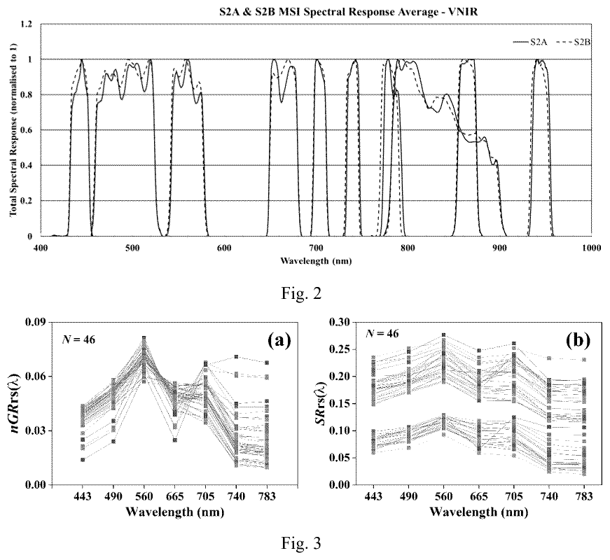

Furthermore, given the differences between satellite multispectral data and hyperspectral proximal sensing reflectance in terms of band position, band width, and wavelength range, and in conjunction with FIG. 2, a method for simulating multispectral data from hyperspectral data is created in Step 3, specifically expressed as:

$$n G R r s ( λ ) = \frac{\int fnR ( λ ) \times S ( λ ) d λ}{\int S ( λ ) d λ} , λ ∈ [ 443 , 490 , 560 , 665 , 705 , 740 , 783 ] ;$$

where fnR(λ) is the preprocessed water body hyperspectral proximal sensing reflectance spectrum data, and S(λ) is a satellite sensor spectral response function at λ nm. Using the aforementioned example and this simulation method, 46 pairs of synchronous data for SRrs(λ) and nGRrs(λ) can be obtained.

Analysis of the 46 pairs of synchronous data for SRrs(λ) and nGRrs(λ) reveals significant differences between SRrs(λ) and nGRrs(λ) at the same band, as shown in FIG. 3. Specifically, (a) and (b) in FIG. 3 are the reflectance spectra of SRrs(λ) and nGRrs(λ) within the 443-783 nm range, respectively.

Therefore, further processing of the preprocessed satellite remote sensing surface reflectance data SRrs(λ) is required. The processing method in this embodiment is as follows:

$$nSRrs ( λ ) = \frac{SRrs ( λ )}{\begin{matrix}( SRrs ( 443 ) + SRrs ( 490 ) + \\ SRrs ( 560 ) + SRrs ( 665 ) + SRrs ( 705 ) + \\ SRrs ( 740 ) + SRrs ( 783 ) ) / 7\end{matrix}} ;$$

wherein λ∈[443, 490, 560, 665, 705, 740, 783], SRrs(443) represents satellite remote sensing surface reflectance data for a band of 443 nm, and likewise for the remaining bands.

Upon comparing the newly obtained processed satellite remote sensing surface reflectance data nSRrs(λ) with the simulation-derived satellite remote sensing surface reflectance nGRrs(λ), analysis indicates that their spectral shapes are essentially consistent.

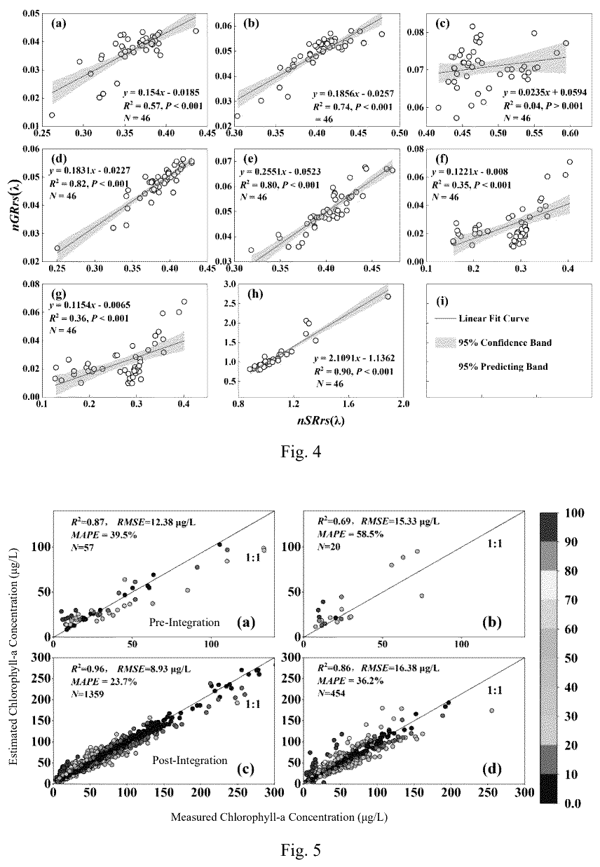

Further, a quantitative conversion relationship between nSRrs(λ) and nGRrs(λ) is constructed band-by-band to eliminate error. The expression of the quantitative conversion relationship between nSRrs(λ) and nGRrs(λ) is as follows:

$$nGRrs ( λ ) = slop \times nSRrs ( λ ) + c ;$$

wherein, slop is a linear regression slope, representing a gain situation between nSRrs(λ) and nGRrs(λ), and c is a constant. In FIG. 4, (a) to (h) are scatter plots of the quantitative conversion relationship between nSRrs(λ) and nGRrs(λ) at 443 nm, 490 nm, 560 nm, 665 nm, 705 nm, 740 nm, and 783 nm, respectively, and (i) in FIG. 4 is an example plot.

Correspondingly, the steps for eliminating the error include: establishing an error analysis principle based on the linear regression slope slop, judging an error situation between nSRrs(λ) and nGRrs(λ) based on the error analysis principle, and performing targeted optimization according to the error situation;
wherein the error analysis principle is:

$$\{ \begin{matrix}slop < 1 & nSRrs ( λ ) is overestimated compared to nGRrs ( λ ) \\ slop = 1 & nSRrs ( λ ) and nGRrs ( λ ) have consistent response \\ slop > 1 & nSRrs ( λ ) is underestimated compared to nGRrs ( λ )\end{matrix} ;$$

and
further optimizing a calibration accuracy of a satellite sensor for water bodies when the overestimation or underestimation occurs; to further illustrate, taking λ∈[443, 490, 560, 665, 705, 740, 783] as an example, the expression of the quantitative conversion relationship for each band is as follows:

$$nGRrs ( λ ) = \begin{matrix}slop \times nSRrs ( λ ) - 0.0185 , λ = 443 , slop = 0.154 , {R}_{λ}^{2} = 0.57 \\ slop \times nSRrs ( λ ) - 0.0257 , λ = 490 , slop = 0.186 , {R}_{λ}^{2} = 0.74 \\ slop \times nSRrs ( λ ) - 0.0594 , λ = 560 , slop = 0.0235 , {R}_{λ}^{2} = 0.04 \\ slop \times nSRrs ( λ ) - 0.0227 λ = 665 , slop = 0.183 , {R}_{λ}^{2} = 0.82 \\ slop \times nSRrs ( λ ) - 0.0523 , λ = 705 , slop = 0225 , {R}_{λ}^{2} = 0.8 \\ slop \times nSRrs ( λ ) - 0.008 , λ = 740 , slop = 0.1221 , {R}_{λ}^{2} = 0.35 \\ slop \times nSRrs ( λ ) - 0.0065 , λ = 783 , slop = 0.115 , {R}_{λ}^{2} = 0.36 \\ slop \times nSRrs ( λ ) - 1.1362 , λ = 705 / 665 , slop = 2.1091 , {R}_{λ}^{2} = 0.9\end{matrix} ;$$

The results show that in this embodiment, the slopes for different bands are all below 1 and not identical, indicating that Sentinel-2 exhibits varying degrees of overestimation across different bands. This may be related to the fact that the official Sentinel-2 atmospheric correction algorithm is developed for land and has insufficient correction for water bodies.

To maximize the accuracy after Sentinel-2 data band conversion, bands with

$${R}_{λ}^{2}$$

higher than 0.5 were selected for conversion, generating a standardized processing flow and method for Sentinel-2 imagery. The included bands are 443 nm, 490 nm, 665 nm, 705 nm, and the 705/665 ratio.

In a further embodiment, the water quality parameter inversion model in Step 5 employs a machine learning model, and the machine learning model comprises: a deep neural network model, a random forest model, a support vector machine model, an extreme gradient boosting tree model, a Gaussian process regression model, and a back-propagation neural network model. To further illustrate, the input features are divided into a training set and a test set in a 7:3 ratio, sequentially input into the machine learning models, and the optimal chlorophyll-a inversion model is selected through comparison. The spectral bands at least include 443 nm, 490 nm, 665 nm, 705 nm, and the 705/665 ratio.

Meanwhile, the following methods can be used to validate the machine learning models, including calculating the model's coefficient of determination, Mean Absolute Percentage Error (MAPE), and Root Mean Square Error (RMSE). Then, the remaining 30% of the synchronous data of SRrs(λ) and water quality and the fused satellite-ground dataset are used as the model validation set, input into the aforementioned constructed models to calculate the coefficient of determination, MAPE, and RMSE. The model with the smallest MAPE and RMSE in both the training dataset and the validation dataset is selected as the inversion model for the water quality parameter chlorophyll-a. Finally, the accuracy validations of the chlorophyll-a models based on the two types of synchronous datasets are compared to evaluate the improvement effect. In this embodiment, the accuracy of the chlorophyll-a model based on the fused satellite-ground dataset is approximately 22.5% higher than that of the chlorophyll-a model based on the synchronous data of SRrs(λ) and water quality, demonstrating that integrating hyperspectral proximal sensing data can significantly improve the accuracy of Sentinel-2 remote sensing inversion for chlorophyll-a, as shown in FIG. 5. Specifically, (a) and (b) in FIG. 5 are scatter plots for constructing and validating the chlorophyll-a inversion model based on the synchronous data of SRrs(λ) and water quality, respectively, and (c) and (d) in FIG. 5 are scatter plots for constructing and validating the chlorophyll-a inversion model based on the fused satellite-ground dataset, respectively.

### Embodiment 2

To implement the method for improving the accuracy of satellite remote sensing inversion by integrating hyperspectral proximal sensing in Embodiment 1, this embodiment discloses a system for improving the accuracy of satellite remote sensing inversion by integrating hyperspectral proximal sensing, wherein the system includes:

a proximal hyperspectral sensor, a satellite sensor, and a water quality detector, configured to acquire a water body hyperspectral proximal sensing reflectance spectrum dataset, a satellite remote sensing surface reflectance dataset, and a water environment in-situ measurement dataset, respectively.

A first module is configured to select corresponding data processing methods to perform data preprocessing on the water body hyperspectral proximal sensing reflectance spectrum dataset, the satellite remote sensing surface reflectance dataset, and the water environment in-situ measurement dataset, respectively.

A second module is configured to determine an optimal matching window by using a temporal matching principle and construct a synchronous dataset; wherein the synchronous dataset comprises: synchronous data of SRrs(λ) and water quality, and synchronous data of fnR(λ) and water quality; and wherein the SRrs(λ) is preprocessed satellite remote sensing surface reflectance data, the fnR(λ) is preprocessed water body hyperspectral proximal sensing reflectance spectrum data, and A is a band.

A third module is configured to create a method for simulating multispectral data from hyperspectral data, generate corresponding synchronous data of nGRrs(λ) and water quality based on the synchronous data of fnR(λ) and water quality, and obtain synchronous data of SRrs(λ) and nGRrs(λ), wherein the nGRrs(λ) is simulation-derived satellite remote sensing surface reflectance.

A fourth module is configured to perform normalization processing on the preprocessed satellite remote sensing surface reflectance data SRrs(λ) based on the synchronous data of SRrs(λ) and nGRrs(λ) to obtain new satellite remote sensing surface reflectance data nSRrs(λ), perform a comparative analysis between the new satellite remote sensing surface reflectance data nSRrs(λ) and the simulation-derived satellite remote sensing surface reflectance nGRrs(λ), and establish a quantitative conversion relationship between nSRrs(λ) and nGRrs(λ) band-by-band to eliminate error.

A fifth module is configured to combine the synchronous data of nGRrs(λ) and water quality and the synchronous data of nSRrs(λ) and water quality to generate a fused satellite-ground dataset; and use the synchronous data of SRrs(λ) and water quality and the fused satellite-ground dataset as input features, respectively, and use the water environment in-situ measurement dataset as an output feature, to construct and train a water quality parameter inversion model.

A sixth module is configured to repeat steps of the third module through the fifth module, update the method for simulating multispectral data from hyperspectral data, a method for enhancing satellite spectral band precision, and the fused satellite-ground dataset in real time, and continuously optimize the water quality parameter inversion model.

A terminal display module is configured to display and download inversion results according to a query instruction until a query termination instruction from a user is received.

Specifically, the system is configured to display and update linear methods for band conversion of satellite data and hyperspectral data, as well as the accuracy of chlorophyll-a inversion models and image results after integrating hyperspectral proximal sensing data. The system is configured to receive user query requests at different temporal scales, statistically analyze and display water quality spatiotemporal change imagery and time-series variation trend characteristics; wherein the different temporal scales at least include four types: daily, monthly, seasonal, and interannual. The system is configured to receive user query requests for different regional scales, statistically analyze and display water quality imagery and time-series variations at the different temporal scales; wherein the different regional scales at least include lake bays and open water areas. The system is configured to receive user download and upload instructions, construct links according to the instructions to download water quality change charts and tables for different temporal scales and regional scales, and continue operation until a query termination instruction is received.

What is claimed is:

1. A method for improving the accuracy of satellite remote sensing inversion by integrating hyperspectral proximal sensing, comprising the steps of:
Step 1: acquiring a water body hyperspectral proximal sensing reflectance spectrum dataset, a satellite remote sensing surface reflectance dataset, and a water environment in-situ measurement dataset, and selecting corresponding data preprocessing methods to perform preprocessing;
Step 2: determining an optimal matching window by using a temporal matching principle and constructing a synchronous dataset; wherein the synchronous dataset comprises: synchronous data of SRrs(λ) and water quality, and synchronous data of fnR(λ) and water quality; wherein the SRrs(λ) is preprocessed satellite remote sensing surface reflectance data, the fnR(λ) is preprocessed water body hyperspectral proximal sensing reflectance spectrum data, and λ is a band; wherein the steps for determining the optimal matching window comprise:
defining a sum of coefficients of determination for all bands as S for screening out an optimal temporal matching window, with the calculation formula being:
$$S = {R}_{443}^{2} + {R}_{490}^{2} + {R}_{560}^{2} + {R}_{665}^{2} + {R}_{705}^{2} + {R}_{740}^{2} + {R}_{783}^{2} ;$$
wherein
$${R}_{443}^{2} , {R}_{490}^{, 2} , {R}_{560}^{2} , {R}_{665}^{2} , {R}_{705}^{2} , {R}_{740}^{2} , {R}_{783}^{2}$$
are coefficients of determination for bands λ∈[443, 490, 560, 665, 705, 740, 783], respectively; and selecting a temporal matching window corresponding to a maximum value of S as the optimal matching window;
Step 3: creating a method for simulating multispectral data from hyperspectral data, generating corresponding synchronous data of nGRrs(λ) and water quality based on the synchronous data of fnR(λ) and water quality, and obtaining synchronous data of SRrs(λ) and nGRrs(λ), wherein the nGRrs(λ) is simulation-derived satellite remote sensing surface reflectance;
Step 4: performing normalization processing on the preprocessed satellite remote sensing surface reflectance data SRrs(λ) based on the synchronous data of SRrs(λ) and nGRrs(λ) to obtain new satellite remote sensing surface reflectance data nSRrs(λ), performing a comparative analysis between the new satellite remote sensing surface reflectance data nSRrs(λ) and the simulation-derived satellite remote sensing surface reflectance nGRrs(λ), and establishing a quantitative conversion relationship between nSRrs(λ) and nGRrs(λ) band-by-band to eliminate error;
Step 5: combining the synchronous data of nGRrs(λ) and water quality and the synchronous data of nSRrs(λ) and water quality to generate a fused satellite-ground dataset; and using the synchronous data of SRrs(λ) and water quality and the fused satellite-ground dataset as input features, respectively, and using the water environment in-situ measurement dataset as an output feature, to construct and train a water quality parameter inversion model; and
Step 6: repeating Step 3 to Step 5, updating the method for simulating multispectral data from hyperspectral data, a method for enhancing satellite spectral band precision, and the fused satellite-ground dataset in real time, continuously optimizing the water quality parameter inversion model, and displaying and downloading inversion results according to a query instruction until a query termination instruction from a user is received.

2. The method for improving the accuracy of satellite remote sensing inversion by integrating hyperspectral proximal sensing according to claim 1, wherein data preprocessing steps for the water body hyperspectral proximal sensing reflectance spectrum dataset comprise:
continuously collecting data under various weather conditions to obtain the water body hyperspectral proximal sensing reflectance spectrum dataset {R(λ)}, and calculating a ratio of a water body hyperspectral proximal sensing reflectance spectrum R(λ) for each band to an average hyperspectral proximal sensing reflectance R₀ within 420 nm to 830 nm, to obtain a normalized hyperspectral proximal sensing reflectance nR(λ) corresponding to the band:
$$nR ( λ ) = \frac{R ( λ )}{{R}_{0}} ;$$
performing radiometric correction on nR(λ) with reference to spectral data of a synchronous terrestrial object spectrum, with the calculation formula being:
$$fnR ( λ ) = α \times nR ( λ ) + b ;$$
where a is a radiometric gain value of a land-based hyperspectral proximal sensor, and b is a radiometric offset value of the land-based hyperspectral proximal sensor.

3. The method for improving the accuracy of satellite remote sensing inversion by integrating hyperspectral proximal sensing according to claim 1, wherein data preprocessing steps for the satellite remote sensing surface reflectance dataset comprise:
acquiring the satellite remote sensing surface reflectance dataset {SR(λ)}, extracting a quality band qa60, and obtaining a cloud mask file pixel value mask_{cloud} according to a bitwise operation principle;
generating an opaque cloud mask file and a cirrus cloud mask file respectively by using binary bitwise operations and the quality band qa60, merging the opaque cloud mask file and the cirrus cloud mask file using an AND( ) function to generate the cloud mask file, thereby obtaining the cloud mask file pixel value mask_{cloud}; and
processing with the following formula to obtain the preprocessed satellite remote sensing surface reflectance data SRrs(λ):
$$SRrs ( λ ) = \frac{SR ( λ )}{π \times 10000} \times {mask}_{cloud} ,$$
where SR(λ) is satellite remote sensing surface reflectance.

4. The method for improving the accuracy of satellite remote sensing inversion by integrating hyperspectral proximal sensing according to claim 1, wherein the method for simulating multispectral data from hyperspectral data is represented by the following formula:
$$nGRrs ( λ ) = \frac{\int fnR ( λ ) \times S ( λ ) d λ}{\int S ( λ ) d λ} , λ ∈ [ 443 , 490 , 560 , 665 , 705 , 740 , 783 ] ;$$
where fnR(λ) is the preprocessed water body hyperspectral proximal sensing reflectance spectrum data, and S(λ) is a satellite sensor spectral response function at λnm.

5. The method for improving the accuracy of satellite remote sensing inversion by integrating hyperspectral proximal sensing according to claim 1, wherein a calculation formula for the normalization processing of the preprocessed satellite remote sensing surface reflectance data SRrs(λ) is as follows:
$$nSRrs ( λ ) = \frac{SRrs ( λ )}{\begin{matrix}( SRrs ( 443 ) + SRrs ( 490 ) + \\ SRrs ( 560 ) + SRrs ( 665 ) + SRrs ( 705 ) + \\ SRrs ( 740 ) + SRrs ( 783 ) ) / 7\end{matrix}} ;$$
wherein λ∈[443, 490, 560, 665, 705, 740, 783], SRrs(443) represents satellite remote sensing surface reflectance data for a band of 443 nm, and likewise for the remaining bands.

6. The method for improving the accuracy of satellite remote sensing inversion by integrating hyperspectral proximal sensing according to claim 1, wherein the quantitative conversion relationship between nSRrs(λ) and nGRrs(λ) is expressed as follows:
$$nGRrs ( λ ) = slop \times nSRrs ( λ ) + c ;$$
wherein, slop is a linear regression slope, representing a gain situation between nSRrs(λ) and nGRrs(λ), and c is a constant.

7. The method for improving the accuracy of satellite remote sensing inversion by integrating hyperspectral proximal sensing according to claim 6, wherein elimination steps for the eliminating error comprise:
establishing an error analysis principle based on the linear regression slope slop, judging an error situation between nSRrs(λ) and nGRrs(λ) based on the error analysis principle, and performing targeted optimization according to the error situation; wherein the error analysis principle is:
$$\{ \begin{matrix}slop < 1 & nSRrs ( λ ) is overestimated compared to nGRrs ( λ ) \\ slop = 1 & nSRrs ( λ ) and nGRrs ( λ ) have consistent response \\ slop > 1 & nSRrs ( λ ) is underestimated compared to nGRrs ( λ )\end{matrix} ;$$
and
further optimizing a calibration accuracy of a satellite sensor for water bodies when the overestimation or underestimation occurs.

8. The method for improving the accuracy of satellite remote sensing inversion by integrating hyperspectral proximal sensing according to claim 1, wherein the water quality parameter inversion model employs a machine learning model, and the machine learning model comprises: a deep neural network model, a random forest model, a support vector machine model, an extreme gradient boosting tree model, a Gaussian process regression model, and a back-propagation neural network model.

9. A system for improving the accuracy of satellite remote sensing inversion by integrating hyperspectral proximal sensing, configured to implement the method for improving the accuracy of satellite remote sensing inversion by integrating hyperspectral proximal sensing according to any one of claims 1 to 8, comprising:
a proximal hyperspectral sensor, a satellite sensor, and a water quality detector, configured to acquire a water body hyperspectral proximal sensing reflectance spectrum dataset, a satellite remote sensing surface reflectance dataset, and a water environment in-situ measurement dataset, respectively;
a first module, configured to select corresponding data processing methods to perform data preprocessing on the water body hyperspectral proximal sensing reflectance spectrum dataset, the satellite remote sensing surface reflectance dataset, and the water environment in-situ measurement dataset, respectively;
a second module, configured to determine an optimal matching window by using a temporal matching principle and construct a synchronous dataset; wherein the synchronous dataset comprises: synchronous data of SRrs(λ) and water quality, and synchronous data of fnR(λ) and water quality; and wherein the SRrs(λ) is preprocessed satellite remote sensing surface reflectance data, the fnR(λ) is preprocessed water body hyperspectral proximal sensing reflectance spectrum data, and λ is a band;
a third module, configured to create a method for simulating multispectral data from hyperspectral data, generate corresponding synchronous data of nGRrs(λ) and water quality based on the synchronous data of fnR(λ) and water quality, and obtain synchronous data of SRrs(λ) and nGRrs(λ), wherein the nGRrs(λ) is simulation-derived satellite remote sensing surface reflectance;
a fourth module, configured to perform normalization processing on the preprocessed satellite remote sensing surface reflectance data SRrs(λ) based on the synchronous data of SRrs(λ) and nGRrs(λ) to obtain new satellite remote sensing surface reflectance data nSRrs(λ), perform a comparative analysis between the new satellite remote sensing surface reflectance data nSRrs(λ) and the simulation-derived satellite remote sensing surface reflectance nGRrs(λ), and establish a quantitative conversion relationship between nSRrs(λ) and nGRrs(λ) band-by-band to eliminate error;
a fifth module, configured to combine the synchronous data of nGRrs(λ) and water quality and the synchronous data of nSRrs(λ) and water quality to generate a fused satellite-ground dataset; and use the synchronous data of SRrs(λ) and water quality and the fused satellite-ground dataset as input features, respectively, and use the water environment in-situ measurement dataset as an output feature, to construct and train a water quality parameter inversion model;
a sixth module, configured to repeat steps of the third module through the fifth module, update the method for simulating multispectral data from hyperspectral data, a method for enhancing satellite spectral band precision, and the fused satellite-ground dataset in real time, and continuously optimize the water quality parameter inversion model; and
a terminal display module, configured to display and download inversion results according to a query instruction until a query termination instruction from a user is received
